(19) United States
(12) Patent Application Publication
(10) Pub. No.: US 2006/0247459 A1
(43) Pub. Date: Nov. 2, 2006

(54) Method for the hydration of a monoolefinically unsaturated compound carrying at least two functional groups

(75) Inventors: Wolfram Stuer, Mannheim (DE); Jens Scheidel, Hirschberg (DE); Peter Bassler, Viernheim (DE)

Correspondence Address: CONNOLLY BOVE LODGE & HUTZ LLP, P.O. BOX 2207, WILMINGTON, DE, 19899-2207, US

(73) Assignee: BASF AKTIENGESELLSCHAFT, Ludwigshafen (DE)

(21) Appl. No.: 10/554,209

(22) PCT Filed: Apr. 21, 2004
(86) PCT No.: PCT/EP04/04205

§ 371 Date: Oct. 24, 2005

(30) Foreign Application Priority Data
Apr. 24, 2003 (DE) 103 18 697.2

(51) Int. Cl. C07F 17/02; C07C 69/02; C07C 253/30

(52) U.S. Cl. 558/459; 560/231; 556/136

(57) ABSTRACT

A process for hydrogenating a monoolefinically unsaturated compound which bears at least two functional groups which are each independently selected from the group consisting of nitrile group, carboxylic acid group, carboxylic ester group, carboxamide group to a saturated compound which bears the same at least two functional groups, in the presence of a rhodium-containing compound, as a catalyst, which is homogeneous with respect to the reaction mixture.

The present invention relates to a process for hydrogenating a monoolefinically unsaturated compound which bears at least two functional groups which are each independently selected from the group consisting of nitrile group, carboxylic acid group, carboxylic ester group and carboxamide group to a saturated compound which bears the same at least two functional groups in the presence of a rhodium-containing compound, as a catalyst, which is homogeneous with respect to the reaction mixture.

Numerous saturated compounds which bear two functional groups which are each independently selected from the group consisting of nitrile group, carboxylic acid group, carboxylic ester group and carboxamide group have great industrial significance. For example, adipic acid or its derivatives are important starting compounds for preparing industrially important polymers such as nylon-6 or nylon-6,6.

Such compounds can be obtained, for example, by adding two terminal olefins which bear the functional groups required to prepare the monoolefinically unsaturated compound containing at least two functional groups.

For instance, hexenedioic diester can be prepared by adding acrylic ester in the presence of appropriate catalyst systems, as described, for example, in J. Organomet. Chem. 1987, 320, C56, U.S. Pat. No. 4,451,665, FR 2,524,341, U.S. Pat. No. 4,889,949, Organometallics, 1986, 5, 1752, J. Mol. Catal. 1993, 85, 149, U.S. Pat. No. 4,594,447, Angew. Chem. Int. Ed. Engl., 1988, 27.185, U.S. Pat. No. 3,013,066, U.S. Pat. No. 4,638,084, EP-A-475 386, JACS 1991, 113, 2777-2779, JACS 1994, 116, 8038-8060.

In such an addition of two terminal olefins which bear the functional groups required to prepare the monoolefinically unsaturated compound containing at least two functional groups, monoolefinically unsaturated compounds are obtained which bear at least two functional groups which are each independently selected from the group consisting of nitrile group, carboxylic acid group, carboxylic ester group and carboxamide group.

It is an object of the present invention to provide a process which enables, in a technically simple and economic manner, the hydrogenation of a monoolefinically unsaturated compound which bears at least two functional groups which are each independently selected from the group consisting of nitrile group, carboxylic acid group, carboxylic ester group and carboxamide group to a saturated compound which bears the same at least two functional groups.

We have found that this object is achieved by the process defined at the outset.

The structures referred to as catalyst in the context of the present invention relate to the compounds which are used as a catalyst; the structures of the catalytically active species under the particular reaction conditions may differ therefrom, but are also included by the term “catalyst” mentioned.

According to the invention, a monoolefinically unsaturated compound which bears at least two functional groups which are each independently selected from the group consisting of nitrile group, carboxylic acid group, carboxylic ester group and carboxamide group is hydrogenated.

In a preferred embodiment, useful monoolefinically unsaturated compounds which bear at least two functional groups which are each independently from the group consisting of nitrile group, carboxylic acid group, carboxylic ester group and carboxamide group are those which are obtainable by adding two terminal olefins which bear the functional groups required to prepare the monoolefinically unsaturated compound containing at least two functional groups.

The terminal olefins used may advantageously be two identical or different, preferably identical, olefins which each independently have the formula H₂C═CHR¹ in which R¹ is a nitrile group, carboxylic acid group, carboxylic ester group or carboxamide group, preferably carboxylic ester group or nitrile group.

In the case of the carboxylic ester group, advantageous compounds are esters of aliphatic, aromatic or heteroaromatic alcohols, in particular aliphatic alcohols. The aliphatic alcohols which can be used are preferably C₁-C₁₀-alkanols, in particular C₁-C₄-alkanols, such as methanol, ethanol, isopropanol, n-propanol, n-butanol, i-butanol, s-butanol, t-butanol, more preferably methanol.

The carboxamide groups may be N— or N,N-substituted, and the N,N-substitution may be identical or different, preferably identical. Useful substituents are preferably aliphatic, aromatic or heteroaromatic substituents, in particular aliphatic substituents, more preferably C₁-C₄-alkyl radicals, such as methyl, ethyl, i-propyl, n-propyl, n-butyl, i-butyl, s-butyl, t-butyl, more preferably methyl.

In an advantageous embodiment, the terminal olefin having a functional group which is used may be acrylic acid or its esters. The preparation of acrylic acid, for example by gas phase oxidation of propene or propane in the presence of heterogeneous catalysts, and the preparation of acrylic esters, for example by esterification of acrylic acid with the appropriate alcohols in the presence of homogeneous catalysts such as p-toluenesulfonic acid are known per se.

When acrylic acid is stored or processed, it is customary to add one or more stabilizers which, for example, prevent or reduce the polymerization or the decomposition of acrylic acid, such as p-methoxyphenol or 4-hydroxy-2,2,4,4-piperidine N-oxide (“4-hydroxy-TEMPO”).

Such stabilizers can be partly or fully removed before the acrylic acid or its esters are used in the addition step. The stabilizer can be removed by processes known per se, such as distillation, extraction or crystallization.

Such stabilizers may remain in the acrylic acid in the amount used beforehand.

When different olefins are used, the addition typically results in mixtures of the different possible addition products.

When one olefin is used, the addition, which in this case is typically referred to as a dimerization, results in one addition product. For economic reasons, this alternative is usually preferred.

In a preferred embodiment, the monoolefinically unsaturated compound which bears at least two functional groups which are each independently selected from the group consisting of nitrile group, carboxylic acid group, carboxylic ester group and carboxamide group is hexenedioic ester, in particular dimethyl hexenedioate, to obtain adipic diester, in particular dimethyl adipate, by hydrogenation.

Adipic acid can be obtained from adipic diester, in particular dimethyl adipate, by cleaving the ester group. Useful processes for this purpose are processes which are for cleaving esters and are known per se.

In a further preferred embodiment, the monoolefinically unsaturated compound which bears at least two functional groups which are each independently selected from the group consisting of nitrile group, carboxylic acid group, carboxylic ester group and carboxamide group is butenedinitrile to obtain adiponitrile by hydrogenation.

In a further preferred embodiment, the monoolefinically unsaturated compound which bears at least two functional groups which are each independently selected from the group consisting of nitrile group, carboxylic acid group, carboxylic ester group, carboxamide group is 5-cyanopentenoic ester, in particular methyl 5-cyanopentenoate, to obtain 5-cyanovaleric ester, in particular methyl 5-cyanovalerate, by hydrogenation.

The addition mentioned of two terminal olefins may be effected by processes known per se, as described, for example, in J. Organomet. Chem. 1987, 320, C56, U.S. Pat. No. 4,451,665, FR 2,524,341, U.S. Pat. No. 4,889,949, Organometallics, 1986, 5, 1752, J. Mol. Catal. 1993, 85, 149, U.S. Pat. No. 4,594,447, Angew. Chem. Int. Ed. Engl., 1988, 27. 185, U.S. Pat. No. 3,013,066, U.S. Pat. No. 4,638,084, EP-A-475 386, JACS 1991, 113, 2777-2779, JACS 1994, 116, 8038-8060.

The addition may advantageously be carried out in the presence of a compound, as a catalyst, which is homogeneous with respect to the reaction mixture and contains rhodium, ruthenium, palladium or nickel, preferably rhodium.

In a preferred embodiment, the addition, in particular dimerization, can be carried out in the presence of the same rhodium-containing compound, as a catalyst, which is homogeneous with respect to the reaction mixture as the hydrogenation in accordance with the process according to the invention of the monoolefinically unsaturated compound obtained by the addition.

In a particularly preferred embodiment, the hydrogenation in accordance with the process according to the invention of the monoolefinically unsaturated compound obtained by the addition may be carried out without removing or depleting the homogeneous, rhodium-containing compound used as a catalyst in the addition, in particular dimerization, of the olefins mentioned.

This procedure is of great advantage compared to the prior art since no workup of the reaction effluent obtained in the addition reaction mentioned is required. In a particularly preferred embodiment, the reaction effluent obtained in the addition reaction, in particular dimerization reaction, can be transferred without a workup step to the hydrogenation in the present process.

This may be effected, for example, by transferring the reaction effluent obtained in the addition reaction from the addition apparatus into a further apparatus intended for the hydrogenation, i.e. by a spatial separation of addition reaction and hydrogenation. For example, the addition reaction may be carried out in a reactor such as a stirred tank, a tank battery such as a stirred tank battery, or a flow tube, or in a combination of one of these reactor types with a further reactor suitable for the hydrogenation.

This may be effected, for example, by carrying out addition reaction and hydrogenation successively in the same apparatus, i.e. a temporal separation of addition reaction and hydrogenation.

Preference is given to carrying out the hydrogenation according to the invention in the presence of a rhodium-containing compound, as a catalyst, which is homogeneous with respect to the reaction mixture and is of the formula [L¹RhL²L³R]⁺X⁻ where
L¹ is an anionic pentahapto ligand, preferably pentamethylcyclopentadienyl;
L² is an uncharged 2-electron donor;
L³ is an uncharged 2-electron donor;
R is selected from the group consisting of H, C₁-C₁₀-alkyl, C₆-C₁₀-aryl and C₇-C₁₀-aralkyl ligands;
X⁻ is a noncoordinating anion, preferably one from the group consisting of BF₄⁻, B(perfluorophenyl)₄⁻, B(3,5-bis(trifluoromethyl)phenyl)₄⁻, Al(OR^{F})₄⁻ where R^{F} is identical or different perfluorinated aliphatic or aromatic radicals, in particular perfluoroisopropyl or perfluoro-tert-butyl;
and where two or three of L², L³ and R are optionally joined.

In a preferred embodiment, L² and L³ may each independently be selected from the group consisting of C₂H₄, CH₂═CHCO₂Me, P(OMe)₃ and MeO₂C—(C₄H₆)—CO₂Me.

In a further preferred embodiment, L² and L³ may be joined together. In this case, L² and L³ together may in particular be acrylonitrile or 5-cyanopentenoic ester.

In a further preferred embodiment, L² and R may be joined together. In this case, L² and R together may in particular be —CH₂—CH₂CO₂Me.

In a further preferred embodiment, L², L³ and R may be joined together. In this case, L², L³ and R together may in particular be MeO₂C(CH₂)₂—(CH)—(CH₂)CO₂Me.

In a particularly preferred embodiment, the hydrogenation may be carried out in the presence of a rhodium-containing compound, as a catalyst, and is homogeneous with respect to the reaction mixture and is selected from the group consisting of

[Cp*Rh(C₂H₄)₂H]⁺ BF₄⁻,

[Cp*Rh(P(OMe)₃)(CH₂═CHCO₂Me)(Me)]⁺ BF₄⁻,

[Cp*Rh(—CH₂—CH₂CO₂Me)(P(OMe)₃)]⁺ BF₄⁻,

[Cp*Rh(MeO₂C(CH₂)₂—(CH—)—(CH₂)CO₂Me)]⁺ BF₄⁻,

[Cp*Rh(C₂H₄)₂H]⁺ B(3,5-bis(trifluoromethyl)phenyl)₄⁻,

[Cp*Rh(P(OMe)₃)(CH₂═CHCO₂Me)(Me)]⁺ B(3,5-bis(trifluoromethyl)phenyl)₄⁻,

[Cp*Rh(—CH₂—CH₂CO₂Me)(P(OMe)₃)]⁺ B(3,5-bis(trifluormethyl)phenyl)₄⁻,

[Cp*Rh(MeO₂C(CH₂)₂—(CH—)—(CH₂)CO₂Me)]⁺ B(3,5-bis(trifluoromethyl)phenyl)₄⁻,

[Cp*Rh(C₂H₄)₂H]⁺ B(perfluorophenyl)₄⁻,

[CP*Rh(P(OMe)₃)(CH₂═CHCO₂Me)(Me)]⁺ B(perfluorophenyl)₄⁻,

[Cp*Rh(—CH₂—CH₂CO₂Me)(P(OMe)₃)]⁺ B(perfluorophenyl)₄⁻ and

[Cp*Rh(MeO₂C(CH₂)₂—(CH—)—(CH₂)CO₂Me)]⁺ B(perfluorophenyl)₄⁻

[Cp*Rh(C₂H₄)₂H]⁺ Al(OR^{F})₄⁻,

[Cp*Rh(P(OMe)₃)(CH₂═CHCO₂Me)(Me)]⁺ Al(OR^{F})₄⁻,

[Cp*Rh(—CH₂—CH₂CO₂Me)(P(OMe)₃)]⁺ Al(OR^{F})₄⁻ and

[Cp*Rh(MeO₂C(CH₂)₂—(CH—)—(CH₂)CO₂Me)]⁺ Al(OR^{F})₄⁻,

where R^{F} is identical or different perfluorinated aliphatic or aromatic radicals, in particular perfluoroisopropyl or perfluoro-tert-butyl.

Such catalysts and their preparation may be effected by processes known per se, as described, for example, in EP-A475 386, JACS 1991, 113, 2777-2779, JACS 1994, 116, 8038-8060.

The hydrogenation of the present invention may advantageously be carried out at a hydrogen partial pressure in the range from 0.1 to 200 bar. In the hydrogenation, an average mean residence time of the monoolefinically unsaturated compound which bears at least two functional groups which are each independently selected from the group consisting of nitrile group, carboxylic acid group, carboxylic ester group, carboxamide group which is in the range from 0.1 to 100 hours has been found to be advantageous. In addition, a useful temperature for the hydrogenation is preferably a temperature in the range from 30° C. to 160° C.

The advantages of the process according to the invention become particularly apparent when at least 5% of the monoolefinically unsaturated compound which bears at least two functional groups which are each independently selected from the group consisting of nitrile group, carboxylic acid group, carboxylic ester group, carboxamide group is hydrogenated to a saturated compound which bears the same as least two functional groups.

### EXAMPLES

Abbreviations used:

Cp* Pentamethylcyclopentadienyl=C₅(CH₃)₅ anion

BAr^{F}₄tetrakis[3,5-bis(trifluoromethyl)phenyl]borate=[B(C₆H₃(CF₃)₂]₄ anion

The experiments were carried out under an atmosphere of dried and repurified argon by means of standard Schlenk techniques. Methylene chloride was dried over P₂O₅; methyl acrylate (Aldrich, stabilized with methoxyphenol) was stored over 4 A molecular sieves and used without further treatment. The Cp*Rh(C₂H₄)₂ complex (Cp*=pentamethylcyclopentadienyl) was prepared starting from [Cp*RhCl₂]₂ by the method of K. Moseley, J. W. Kang, P. M. Maitlis J. Chem. Soc. (A) 1970, 2875-2883. The starting material [Cp*RhCl₂]₂ was synthesized by the method of B. L. Booth, R. N. Hazeldine, M. Hill J. Chem. Soc. (A) 1969, 1299-1303.

The acid required to activate the catalyst, HBAr^{F}₄, was prepared according to M. Brookhart, B. Grant, A. F. Volpe Organometallics 1992, 11, 3920-3922. In this context, HBAr^{F}₄ refers to the bis-etherate of tetrakis[3,5-bis(trifluoromethyl)phenyl]boric acid.

The reaction effluents were analyzed by means of GC (instrument: Hewlett Packard 5820; column: HP-5; length: 30 m; diameter: 0.25 mm; film thickness: 1.0 μm), and the structures of the products had been elucidated beforehand by means of GC-MS coupling. All data are in area percent.

### Example 1

In a similar manner to Example 14 in EP 475 386, 20 mg (0.068 mmol) of Cp*Rh(C₂H₄)₂ were admixed in a suitable reaction vessel first with 40 ml of methyl acrylate and then, at 0° C., with a solution of the stoichiometric amount (based on Rh) of the acid HBAr^{F}₄ in 10 ml of CH₂Cl₂. The mixture was heated to 55° C. and, after certain times, samples were taken for gas chromatography investigation (see Table 1).

Without hydrogen addition, the reaction came to a standstill after only 2 h. After 22 h, the protective gas atmosphere was exchanged for hydrogen (1 bar) by opening the line to the hydrogen supply and allowing further hydrogen to flow in as required. The progress of the dimerization was then observed (24 h). Up to this time, dimethyl adipate had not been detected. After 90 h, the linear dimeric esters had been almost fully hydrogenated to dimethyl adipate. At this time, another 40 ml of methyl acrylate were added. The sample taken immediately after the addition (time still=90 h) allows dilution of the reaction effluent by methyl acrylate to be observed. After a further 2 h, the unsaturated dimerization products of the formula MeOOC-(n-C₄H₆)—COOMe could again be observed. The proportion missing from 100% consists of methylene chloride and also small amounts of methyl propionate, branched dimers and trimers. The example confirms that the catalyst is still active in the dimerization of methyl acrylate even after the hydrogenation.

TABLE 1

| | Methyl acrylate | Lin. unsaturated diesters | Dimethyl adipate |
|---|---|---|---|
| Time | H₂C═CH—CO₂Me | MeO₂C-(nC₄H₆)—CO₂Me | MeO₂C-(nC₄H₈)—CO₂Me |
| [h] | [area %] | [area %] | [area %] |
| 2 | 63.7 | 26.8 | 0.0 |
| 4 | 63.3 | 27.5 | 0.0 |
| 22 | 63.4 | 27.5 | 0.0 |
| 24 | 8.6 | 82.9 | 0.0 |
| 90 | 0.0 | 2.8 | 87.5 |
| 90 | 55.9 | 1.3 | 39.1 |
| 93 | 29.2 | 23.8 | 40.8 |

A comparative experiment in which operation was effected from the start with the hydrogen line open confirms that an experimental phase without hydrogen feed is not required.

### Example 2

In a similar manner to Example 1, 60 mg (0.204 mmol) of Cp*Rh(C₂H₄)₂ were admixed in a suitable reaction vessel first with 120 ml of methyl acrylate and then, at room temperature, with a stoichiometric amount (based on Rh) of the acid HBAr^{F}₄. 500 ppm of phenothiazine were added to the mixture as a polymerization inhibitor. The mixture was heated to 80° C. and stirred with a sparging stirrer under 1 bar of hydrogen. After 53 h, the pressure was increased from 1 bar to 5 bar of H₂. After certain times, samples were taken for gas chromatography investigation (see Table 2). The proportion missing from 100% consists of methyl propionate and also small amounts of branched dimers and trimers.

The example shows that the reaction can also be carried out at 80° C., without solvents and in the presence of a further polymerization inhibitor (in this case phenothiazine).

TABLE 2

| | | Lin. unsaturated | |
|---|---|---|---|
| | H₂ | diesters | Dimethyl adipate |
| Time | pressure | MeO₂C-(nC₄H₆)—CO₂Me | MeO₂C-(nC₄H₈)—CO₂Me |
| [h] | [bar] | [area %] | [area %] |
| 21 | 1 | 75.1 | 18.5 |
| 28.5 | 1 | 70.6 | 23.0 |
| 45 | 1 | 66.0 | 28.1 |
| 53 | 5 | 64.1 | 29.8 |
| 69.5 | 5 | 61.8 | 32.0 |
| 142.5 | 5 | 54.0 | 40.2 |

### Example 3

In a similar manner to Example 1, 60 mg (0.204 mmol) of Cp*Rh(C₂H₄)₂ were admixed in a suitable reaction vessel first with 120 ml of methyl acrylate and then, at room temperature, with a stoichiometric amount (based on Rh) of the acid HBAr^{F}₄. The mixture was heated to 80° C. and stirred with a sparging stirrer under 1 bar of hydrogen. After certain times, samples were taken for gas chromatography investigation (see Table 3).

The proportion missing from 100% consists of methyl propionate and also small amounts of branched dimers and trimers.

The example shows that the reaction can also be carried out at 80° C. without polymerization inhibitor.

TABLE 3

| | Methyl acrylate | Lin. unsaturated diesters | Dimethyl adipate |
|---|---|---|---|
| Time | H₂C═CH—CO₂Me | MeO₂C-(nC₄H₆)—CO₂Me | MeO₂C-(nC₄H₈)—CO₂Me |
| [h] | [area %] | [area %] | [area %] |
| 21 | 0.4 | 69.3 | 23.5 |
| 45 | 0.0 | 44.6 | 48.4 |
| 117.5 | 0.0 | 6.7 | 86.9 |

1. A process for hydrogenating a monoolefinically unsaturated compound which bears at least two functional groups which are each independently selected from the group consisting of nitrile group, carboxylic acid group, carboxylic ester group and carboxamide group to a saturated compound which bears the same at least two functional groups, in the presence of a rhodium-containing compound, as a catalyst, which is homogeneous with respect to the reaction mixture.

2. A process as claimed in claim 1, wherein the monoolefinically unsaturated compound used is a compound which is obtainable by adding two terminal olefins which bear the functional groups required to prepare the monoolefinically unsaturated compound containing at least two functional groups.

3. A process as claimed in claim 2, wherein the terminal olefins used are two olefins which each independently have the formula H₂C═CHR¹ in which R¹ is a nitrile group, carboxylic acid group, carboxylic ester group and carboxamide group.

4. A process as claimed in claim 2, wherein the addition is carried out in the presence of a compound, as a catalyst, which is homogeneous with respect to the reaction mixture and contains rhodium, ruthenium, palladium or nickel.

5. A process as claimed in claim 2, wherein the addition is carried out in the presence of a compound, as a catalyst, which is homogeneous with respect to the reaction mixture and contains rhodium.

6. A process as claimed in claim 1, wherein the same rhodium-containing compound is used as a catalyst in the addition.

7. A process as claimed in claim 1, wherein the monoolefinically unsaturated compound which bears at least two functional groups which are each independently selected from the group consisting of nitrile group and carboxylic acid group is carboxylic ester group and carboxamide group is hexenedioic diester to obtain adipic diester in the hydrogenation.

8. A process as claimed in claim 1, wherein the monoolefinically unsaturated compound which bears at least two functional groups which are each independently selected from the group consisting of nitrile group, carboxylic acid group, carboxylic ester group and carboxamide group is butenedinitrile to obtain adipodinitrile in the hydrogenation.

9. A process as claimed in claim 1, wherein the monoolefinically unsaturated compound which bears at least two functional groups which are each independently selected from the group consisting of nitrile group, carboxylic acid group, carboxylic ester group and carboxamide group is 5-cyanopentenoic ester to obtain 5-cyanovaleric ester in the hydrogenation.

10. A process as claimed in claim 1, wherein the hydrogenation is carried out in the presence of a rhodium-containing compound, as a catalyst, which is homogeneous with respect to the reaction mixture and has the formula [L¹RhL²L³R]⁺X⁻ where
L¹ is an anionic pentahapto ligand;
L² is an uncharged 2-electron donor;
L³ is an uncharged 2-electron donor;
R is selected from the group consisting of H, C₁-C₁₀-alkyl, C₆-C₁₀-aryl and C₇-C₁₀-aralkyl ligands;
X⁻ is a noncoordinating anion;
and where two or three of L², L³ and R are optionally joined.

11. A process as claimed in claim 10, wherein L¹ is pentamethylcyclopentadienyl.

12. A process as claimed in claim 10, wherein X⁻ is selected from the group consisting of BF₄⁻, B(perfluorophenyl)₄⁻, B(3,5-bis(trifluoromethyl)phenyl)₄⁻, Al(OR^{F})₄⁻ where R^{F} is identical or different perfluorinated aliphatic or aromatic radicals.

13. A process as claimed in claim 10, wherein L² and L³ are each independently selected from the group consisting of C₂H₄, CH₂═CHCO₂Me, P(OMe)₃ and MeO₂C—(C₄H₆)—CO₂Me.

14. A process as claimed in claim 10, wherein L² and L³ together are selected from the group consisting of acrylonitrile and 5-cyanopentenoic ester.

15. A process as claimed in claim 10, wherein L² and R together are —CH₂—CH₂CO₂Me.

16. A process as claimed in claim 10, wherein L², L³ and R together are MeO₂C(CH₂)₂—(CH)—(CH₂)CO₂Me.

17. A process as claimed in claim 10, wherein the hydrogenation is carried out in the presence of a rhodium-containing compound, as a catalyst, which is homogeneous with respect to the reaction mixture and is selected from the group consisting of
[Cp*Rh(C₂H₄)₂H]⁺ BF₄⁻,
[Cp*Rh(P(OMe)₃)(CH₂═CHCO₂Me)(Me)]⁺ BF₄⁻,
[Cp*Rh(—CH₂—CH₂CO₂Me)(P(OMe)₃)]⁺ BF₄⁻,
[Cp*Rh(MeO₂C(CH₂)₂—(CH—)—(CH₂)CO₂Me)]⁺ BF₄⁻,
[Cp*Rh(C₂H₄)₂H]⁺ B(3,5-bis(trifluoromethyl)phenyl)₄⁻,
[Cp*Rh(P(OMe)₃)(CH₂═CHCO₂Me)(Me)]⁺ B(3,5-bis(trifluoromethyl)phenyl)₄⁻,
[Cp*Rh(—CH₂—CH₂CO₂Me)(P(OMe)₃)]⁺ B(3,5-bis(trifluormethyl)phenyl)₄⁻,
[Cp*Rh(MeO₂C(CH₂)₂—(CH—)—(CH₂)CO₂Me)]⁺ B(3,5-bis(trifluoromethyl)phenyl)₄⁻,
[Cp*Rh(C₂H₄)₂H]⁺ B(perfluorophenyl)₄⁻,
[Cp*Rh(P(OMe)₃)(CH₂═CHCO₂Me)(Me)]⁺ B(perfluorophenyl)₄⁻,
[Cp*Rh(—CH₂—CH₂CO₂Me)(P(OMe)₃)]⁺ B(perfluorophenyl)₄⁻,
[Cp*Rh(MeO₂C(CH₂)₂—(CH—)—(CH₂)CO₂Me)]⁺ B(perfluorophenyl)₄⁻,
[Cp*Rh(C₂H₄)₂H]⁺ Al(OR^{F})₄⁻,
[Cp*Rh(P(OMe)₃)(CH₂═CHCO₂Me)(Me)]⁺ Al(OR^{F})₄⁻,
[Cp*Rh(—CH₂—CH₂CO₂Me)(P(OMe)₃)]⁺ Al(OR^{F})₄⁻ and
[Cp*Rh(MeO₂C(CH₂)₂—(CH—)—(CH₂)CO₂Me)]⁺ Al(OR^{F})₄⁻,
where R^{F} is identical or different perfluorinated aliphatic or aromatic radicals.

18. A process as claimed in claim 1, wherein the hydrogenation is carried out at a partial hydrogen pressure in the range from 0.1 bar to 200 bar.

19. A process as claimed in claim 1, wherein the hydrogenation is carried out at an average mean residence time of the monoolefinically unsaturated compound which bears at least two functional groups which are each independently selected from the group consisting of nitrile group, carboxylic acid group, carboxylic ester group and carboxamide group which is in the range from 0.1 to 100 hours.

20. A process as claimed in claim 1, wherein the hydrogenation is carried out at a temperature in the range from 30° C. to 160° C.

21. A process as claimed in claim 1, wherein at least 5% of the monoolefinically unsaturated compound which bears at least two functional groups which are each independently selected from the group consisting of nitrile group, carboxylic acid group, carboxylic ester group and carboxamide group is hydrogenated to a saturated compound which bears the same at least two functional groups.

22. A process as claimed in claim 5, wherein the mixture obtained in the addition is fed to a hydrogenation as claimed in claim 1 without removing the rhodium-containing compound used as a catalyst.
